(19) United States
(12) Patent Application Publication
(10) Pub. No.: US 2003/0036098 A1
(43) Pub. Date: Feb. 20, 2003

(54) Axial pattern analysis utilizing organisms having defined marker patterns

(76) Inventors: Anthony A. Ferrante, Belmont, MA (US); W. Peter Hansen, Canaan, NY (US); Petra B. Krauledat, Canaan, NY (US)

Correspondence Address: Brenda Herschbach Jarrell, Ph.D., Choate, Hall & Stewart, Exchange Place, 53 State Street, Boston, MA, 02109, US

(21) Appl. No.: 09/932,413

(22) Filed: Aug. 17, 2001

(51) Int. Cl. A01K 67/033; G01N 33/00; G01N 33/53; G01N 33/567

(52) U.S. Cl. 435/7.2; 800/13; 702/21

(57) ABSTRACT

A method of using elongate multicellular organisms in conjunction with a specialized flow cytometer for drug discovery and compound screening. A stable, optically detectable linear marker pattern on each organism is used to construct a longitudinal map of each organism as it passes through the analysis region of the flow cytometer. This pattern is used to limit complex data analysis to particular regions of each organism thereby simplifying and speeding analysis. The longitudinal marker pattern can be used to alter signal detection modes at known regions of the organism to enhance sensitivity and overall detection effectiveness. A repeating pattern can also be used to add a synchronous element to data analysis. The marker patterns are established using known methods of molecular biology to express various indicator molecules. Inherent features of the organism can be rendered detectable to serve as marker patterns.

### BACKGROUND OF THE INVENTION

Area of the Art

This invention concerns the field of automated analysis of complex, multicellular model organisms that are particularly useful in the field of drug discovery and in the field of toxicology.

### DESCRIPTION OF THE PRIOR ART AND SUMMARY OF THE INVENTION

Drug discovery assays have been developed for a variety of biochemical pathways in vitro. Each assay generally works on only one step of an often-complex pathway. An assay can be designed in a complex, living organism such that a compound that affects any component of a biochemical pathway could be identified as a “hit”. In addition, use of a complex organism can also provide data relating to toxicity and impact of the compound on other biochemical pathways thus yielding more relevant information.

Fluorescent protein genes have been used as reporters for gene expression in a wide variety of organisms (Tsien, Nature Biotechnology). The present invention permanently incorporates fluorescent proteins into multicellular organisms to create spatially marked strains that can be used in combination with a high-speed flow cytometer to detect and map the spatial location of other, experimentally generated gene expression in large populations of organisms with a high degree of precision. The marker patterns serve as guides to focus and synchronize the signal processing and computational electronics on specific spatial regions of the experimental organism where expression is expected, thus improving processing speed and accuracy. In addition, the location of experimental gene expression is mapped by reference to the invariant, spatial positions of the fluorescent proteins in the marked strain thus providing clues about the developmental aspects of the expression event.

The essential characteristics of the marker strains are described in terms of the nematode Caenorhabditis elegans by way of example. The co-pending U.S. patent application Ser. No. 09/465,215 describes a method for orienting, analyzing and sorting large, elongate, multicellular, organisms in a modified flow cytometer. The contents of that application are specifically incorporated herein by reference. In that inventive device nematodes flow through the disclosed apparatus, one-by-one, with their long axes substantially oriented along the direction of flow. Each organism flows through a light beam that is focused to a narrow line substantially perpendicular to the long axis of the nematode. Typically, the line focus is of the order of 0.5 millimeter to 2 millimeters in the dimension perpendicular to the long axis (i.e., width of the focused line) of the organism, and 20 micrometers or less in the dimension along the axis of the organism (i.e., height of the focused line). As a consequence of this narrow line focus, only a small fraction of the entire length of the organism is illuminated at any given moment while it flows through the light beam. Light scatter and fluorescence detectors are arranged at various positions to collect light that is scattered and fluorescence that is emitted as the nematode passes through the light beam.

The temporal signal generated by any of these detectors corresponds to a spatial profile of light scattering regions or fluorescence generating regions along the long axis of the organism. Methods of using narrowly focused laser beams to create a profile of light scatter and fluorescence have been reported in flow cytometry applications by Wheeless and others (See, Flow Cytometry and Sorting, Second edition, 1990, Wiley-Liss, Inc.). These methods have come to be termed “slit-scanning”. Slit-scanning has been directed to ascertaining nuclear size and shape in single cells, nuclear/cell-diameter ratios, identification of single, multinucleated cells, chromosome shape features including chromosome length and centromeric index, and head-shape measurements in sperm. It has also been used to identify and estimate the size of colonies of phytoplankton.

Methods of detecting fine detail in slit-scanning have relied on apparatus such as diffraction limited optics to create a narrow line focus and image plane masks to act as optical spatial filters. With the disclosed apparatus, optical resolution of details as small as 0.8 micrometers has been achieved along the flow axis dimension of the object being scanned. Prior art instrumentation performed a slit scan of whole organisms as they passed through the analysis zone of the laser, or, in the instrumentation described by Byerly, through a Coulter orifice.

What has not been provided previously is a method for accurately describing the position of an experimental feature relative to other invariant features in the axial scan. Locating features in a multicellular organism is an important tool for understanding development and differentiation of structures during the organism's life cycle. A need for this tool is acutely felt in the area of drug discovery where multicellular organisms are used as disease models. The problem of high-speed analysis of placement of axial features within transparent, or partially transparent, multicellular organisms, is solved by the present invention, thus providing a much needed tool for developmental biology and drug discovery.

When analyzed by slit-scanning, multicellular organisms present more complex background profiles of light scatter and autofluorescence than do single cells. It is against this complex background that features, such as fluorescent protein expression must be detected, and spatially located. The diameter of a mature C. elegans is approximately 70 micrometers. All things being equal, this means that the background autofluorescence from a nematode is approximately ten times that from a white blood cell (about seven micrometers in diameter), while a fluorescence reporter signal from a single C. elegans cell is no greater than that from a single blood cell. In the case of Drosophila melanogaster (fruit fly) larvae, the situation is even worse because the diameter of an advanced stage larva is of the order of one millimeter, which means that autofluorescence is much more than a hundred times greater than in single blood cells. This means that experimentally created, fluorescent features along the axis of a multicellular organism may produce a much weaker optical signal than the autofluorescence background. One can imagine an axial profile of autofluorescence with very high peaks and valleys effectively masking an experimentally created fluorescence feature. The present invention provides the means to locate and measure the intensity of experimentally created optical features in the presence of overwhelming autofluorescence.

The present invention employs strong fluorescence markers that can be detected against the strong autofluorescence background and used to “bracket” a section of the signal (i.e., a specific lengthwise region of the organism) where the experimentally created feature is expected to appear and electronically process only this smaller amount of electronic data. This shortened processing task provides valuable processing time for other tasks such as commanding a sorter mechanism before the organism has time to flow beyond the sorter's deflection point. To obtain adequate resolution of axial features the height of the line focus beam must be substantially smaller than the length of the organism analyzed. In addition, the invention provides a means to reduce the variability of the autofluorescence profile and improve the detection of the markers.

The cells of multicellular organisms like C. elegans and Drosophila melanogaster (unlike phytoplankton) develop in a reproducible and spatially organized way. This organization is governed by both intracellular and intercellular interactions that provide developing cells with “positional information.” Since the spatial location of cells is highly conserved from generation to generation in species such as the fly D. melanogaster, and the nematode C. elegans, it is possible to express markers that will become stable features of a particular genetic strain of the organism. The present invention takes advantage of this to provide a high signal-to-background “map” of invariant locations along the length of the organism. These locations serve as positional markers to bracket and isolate segments of an axial profile for signal processing.

A strain exhibiting such a “map” (marker pattern) can then be used in a number of research protocols where experimental fluorescence markers are created in a pattern that is independent of the strain marker pattern. The strain marker pattern serves as a reference for the spatial position of the experimentally induced fluorescence markers. Further, the synchronous nature of the markers wherein a marker signal will be found at an expected point allows enhanced detection of the marker signals against background noise.

An example of establishing a fluorescent marker strain of C. elegans follows. The genetic manipulations described are well known to those of skill in the art. The invention comprises the use of these genetic constructs. First, one constructs an expression vector that carries a gene for a fluorescent protein, for example ZsYellow from ClonTech, Inc., under control of the egl-17 promoter sequence. Next, insertion of this construct into the organism's genome results in expression in the M4 neuron located in the anterior bulb of the pharynx. This expression commences soon after the organism hatches and persists through adulthood. This insertion also results in expression in vulval precursor cells as early as the late L2 larval stage and continuing at lower levels in the vulva of the adult hermaphrodite worm. This pattern of fluorescence expression will be carried as a stable characteristic of the strain resulting from the insertion of the construct into the genome. The fluorescent signal in the head provides the instrument with a means to determine the orientation of the animal while the signal at the vulva provides additional positional information as well as providing some information regarding developmental stage. The invention consists of producing a stable longitudinal pattern and using it as an aid in signal processing. A preferred method is to construct a strain of organism with stable fluorescent markers. The variety of promoters and other genetic constructs that can be employed to achieve this aim is almost limitless.

Generally, the marker fluorescence pattern and the experimentally induced fluorescence pattern will be detectable by different optical channels. That is, if the marker pattern is one of red fluorescence, it is often advantageous to design the experimental treatment (e.g., a screen of potential pharmaceuticals) to show function by producing localized green fluorescence (i.e., non-red fluorescence). In such a scheme, the instrument can be instructed to look for a specific optical pattern using the red fluorescence optics to determine the longitudinal orientation of the organism and to provide additional positional information. Because this signal pattern can be preprogrammed, analysis can be performed more rapidly than if a more complex and variable single color optical system were used. The instrument then compares features in the green fluorescence signal to the positional information in the red. This approach has the further advantage that if the various features of the organism are closely spaced they are more easily resolved if multiple fluorescence markers are used. In some cases a third or even more channels (colors) can be used. Alternatively, it is possible to use only a single optical and electronic channel for both patterns (marker pattern and experimental or test). This would be useful in a case in which a version of the instrument described in patent application Ser. No. 09/465,215 was employed that utilized only one set of fluorescence optics. It is simply a matter of balancing instrument complexity and cost against the value of the added information obtained.

The point of the invention is a detectable spatial pattern used for improving signal processing and generally serving as a “map” to pinpoint the location of detectable patterns created or altered by experimental treatments. This does not necessarily require that the genetic manipulation be used to directly create a fluorescent marker pattern. Exogenous markers such as fluorescently labeled lectins, particles or antibodies can also be used to mark the location of features created by genetic manipulation or of existing structures, such as the vulva, to create a pattern useful for signal processing. That is, the created spatial pattern may not be optically detectable until after treatment with a ligand or with a histochemical process. For example, the promoter or other spatially oriented genetic control element may actually control local expression of an enzyme whose presence is made detectable by a histochemical procedure prior to flow cytometric analysis of the organisms. The detection may be by means of fluorescence or by light absorption or light scatter. Light absorption or scatter may be due to a ligand, a histochemically synthesized dye or compound (e.g. precipitation product of a histochemical such as diaminobenzidine or a tetrazolium salt). Also, a particularly dense deposit of a protein or other biomolecule or structure resulting from the genetic manipulation may also be detectable by light scatter or other optical methods. In some cases there may be a useful “inherent” or “latent” pattern within a strain of test organisms. In that case treatment with a lectin or antibody is all that is needed to make the pattern usable.

Since not all markers can be made arbitrarily strong, a means to reduce the effects of autofluorescence is also important. Organisms are not oriented in an azimuthal direction in this invention, but are oriented only along the axis of flow. Consequently, different cellular masses are stimulated into autofluorescence depending upon the azimuthal orientation with respect to the laser axis. In other words, there will be differences in the autofluorescence profile for each organism that passes through the laser because each organism will be in a different azimuthal orientation (e.g., vulva toward laser or vulva turned away from laser). To compensate for this, a second wavelength band of autofluorescence that lies outside the experimentally created fluorescence band can be monitored, and subtracted from the total profile. Azimuthal variations in autofluorescence in the two different bands will correlate. Subtraction of the second wavelength band of autofluorescence decreases autofluorescence without significantly altering the measured fluorescence signal from the experimentally created marker. Subtraction reduces the variability in the autofluorescence profile from organism to organism.

Signal processing electronics can be configured to integrate fluorescence signals or to detect the peak of such signals. Integration is useful in reducing electronic noise or laser noise for a spatially diffuse feature, and peak detection is useful in pinpointing the location of a spatially sharp feature. A marker strain profile can be used to trigger different signal processing methods (e.g. integration or peak detection) depending on the nature of the experimentally created feature. For example, a given marker strain might produce five spaced-apart marker features along the length of the organism. These marker features are reasonably strong so that peak detection would work well. However, the experimentally induced marker appears between the third and the fourth marker and is fairly diffuse spatially. Therefore, the system could advantageously be programmed to switch from peak detection to integration after the third marker is detected. This would allow optimal detection of the experimentally induced marker. It is only with the use of the tailored marker pattern strains of the present invention that such switching of signal processing electronics becomes possible.

### BRIEF DESCRIPTION OF THE FIGURES

FIG. 1 shows a series of photomicrographs of transgenic C. elegans expressing ZsYellow under the control of the egl-17 promoter with FIG. 1A showing a white light image with the corresponding fluorescence image shown in FIG. 1C; FIG. 1B similarly corresponds to the fluorescence image of FIG. 1D.

FIG. 2 shows oscilloscope tracings of optical detector signals resulting from flow cytometric analysis of C. elegans with FIG. 2A and FIG. 2B showing transgenic egl-17 expressing organisms corresponding to the organisms of FIG. 1 and FIG. 2C showing wild type C. elegans as a control;

FIG. 3 shows PY1089 transgenic organisms expressing GFP (and showing autofluorescence) with FIG. 3A showing a light micrograph and FIG. 3B showing the corresponding fluorescence micrograph while FIGS. 3C and 3D show optical detector signals from these organisms undergoing flow analysis; and

FIG. 4 shows optical detector signals that result from flow cytometric analysis of transgenic organisms resulting from mating the egl-17::Zs Yellow construct into PY1089.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide improved data processing of optical signals from elongate multicellular organisms by use of a pattern of markers of spaced apart along the long axis of the organisms.

Creating A Marker Strain

A general approach to creating a marker strain of organisms is to genetically introduce a set of features that are readily detected by a flow cytometer. A simple approach is to produce features that can be directly detected by their fluorescence-for example by introducing a gene for a fluorescent protein. Any detectable pattern can be used, however. Enzyme patterns can be detected by histochemical reactions producing a colored or fluorescent product. Proteins can be overexpressed so as to be optically detectable. Other biological products such as fat globules, crystals or natural pigments can also serve to form an optically detectable pattern. The pattern could be antigenic and be detected by of antibodies, or the pattern could be carbohydrate-based and detectable by addition of lectins. The lectins and antibodies can be fluorescent, or can be linked to histochemically detectable molecules or optically detectable structures such as microspheres. Although in most instances it will be necessary to employ genetic manipulation to produce an optimal marker strain, some naturally occurring organisms or strains of organisms have cryptic marker features that can be revealed through the application or antibodies, histochemicals or other such methods.

In the case of genetic manipulation it is advantageous to select a promoter that will result in a desired spatial pattern of expression. An example of such a promoter is the egl-17 promoter of C. elegans. This promoter sequence, when inserted at the 5′ end of a gene, will result in expression of the gene product (protein) in the M4 neuron and in vulval precursor cells of the organism. The positions of these cells are well characterized and are invariant in a wild-type (N2) background.

The gene controlled by the chosen promoter should encode a detectable product. An example of such a gene product is a fluorescent protein such as the AsRed gene (ClonTech, Inc.). As already mentioned, a large variety of other detection methods are available such as those involving enzymatic or antigenic properties. An advantage of a fluorescent protein is that the organism can be analyzed directly with no need for special incubations or other sample preparation.

Standard molecular genetic techniques are used to clone the promoter DNA sequence, the detectable protein gene sequence, and other DNA sequences required for optimal expression in the organism into an appropriate plasmid vector. For example, the present inventors and their associates have constructed a series of expression vectors in which a synthetic intron has been inserted at the 5′ end and the C. elegans unc-54, a 3′ UTR (untranslated region), has been inserted at the 3′ end of each ClonTech Reef Coral Protein gene (AmCyan, ZsGreen, ZsYellow, DsRed, DsRed2, or AsRed). The egl-17 promoter sequence has been inserted upstream of the 5′ synthetic intron in each of the expression vectors resulting in egl-17 expression plasmid constructs for each fluorescent protein.

The expression plasmid DNA is then inserted into the genome of the host organism. One method used for C. elegans entails microinjecting plasmid DNA into the gonad of young adult hermaphrodites and selecting progeny that express the detectable marker. Such animals generally carry the marker DNA as an unstable extrachromosomal array. Additional steps are required to cause the DNA to become integrated into a chromosome and to select the progeny bearing this integration. This is generally accomplished by mutagenizing the animals to introduce random double stranded breaks in chromosomal DNA. During the DNA repair process extrachromosomal sequences can become incorporated into a chromosome. F₂ progeny that have undergone such an incorporation event can be screened. F₂ homozygotes from such an integration event are identified based on their ability to transfer the marker DNA to 100 percent of their progeny. It should be noted that other methods, including some that result in integration into a specific site in a chromosome can also be used. The point of the present invention is use of the pattern-marked organism as opposed to creation of such an organism.

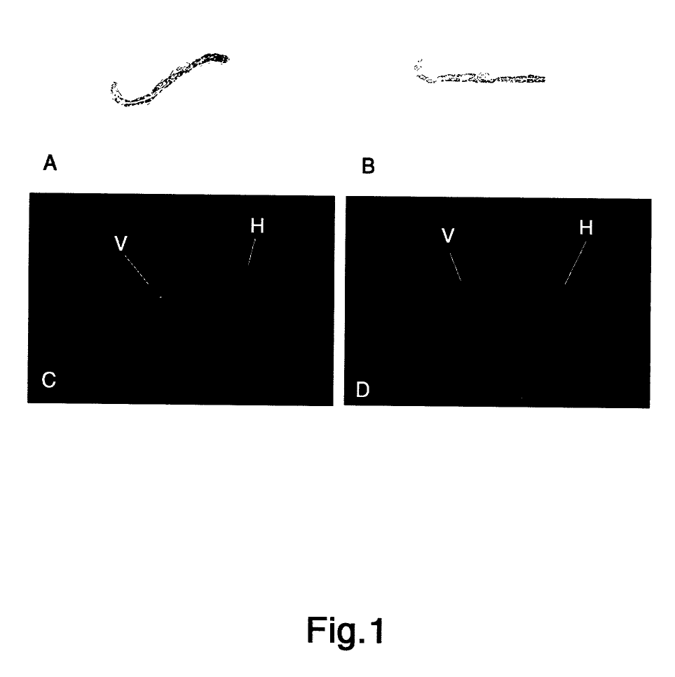

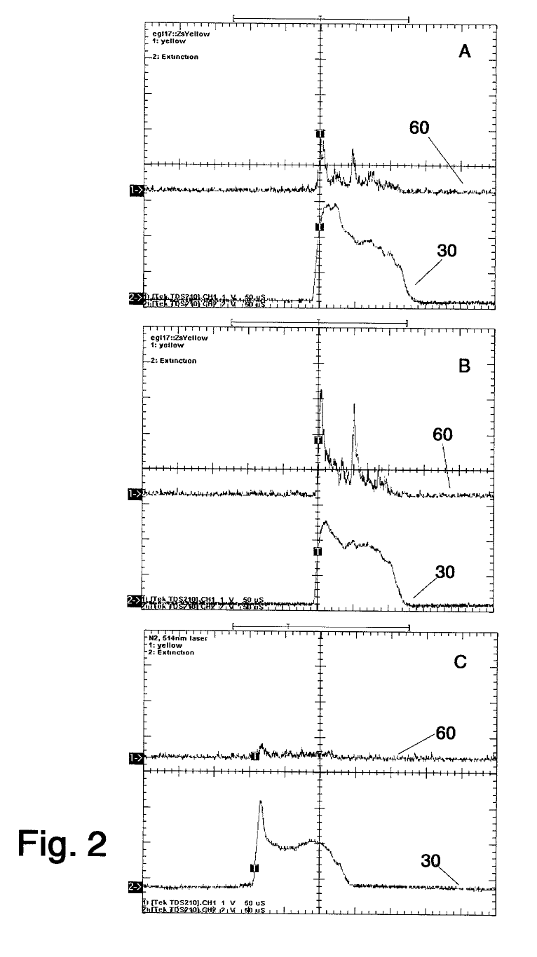

FIG. 1 shows photomicrographs of transgenic organisms where expression of a fluorescent protein is under the control of the egl-17 promoter. In this case the construct is egl-17::ZsYellow. FIGS. 1A and 1B show light micrographs of two organisms with FIGS. 1C and 1D showing the corresponding fluorescence images with the head (H) and vulva (V) marked. Diffuse autofluorescence of the gut is discernible between the head and vulva. FIG. 2 shows oscilloscope traces of the optical detector signal from flow cytometric analysis of these organisms. FIGS. 2A and 2B show oscilloscope traces from representative egl-17::Zs Yellow expressing C. elegans. An extinction signal 30 indicates when the organism enters and exits the laser beam. In the above-referenced co-pending application it is disclosed that forward light scatter measured over a wide solid angle is often a more effective discriminator than simple extinction. It should be understood that all references herein to extinction can be replaced by wide-angle forward light scatter or another signal shown to effectively indicate presence of an organism. The upper trace 60 represents yellow fluorescence. The yellow fluorescence signal 60 is indicative of the presence of ZsYellow and marks the head and vulva in the organism. These precisely located points of fluorescence represent a marker pattern as used in the present invention. These results should be compared with the trace (FIG. 2C) of a control organism lacking the genetic construct.

In cases where mutagenesis has been employed, it is advantageous to remove extraneous mutations by performing several rounds of mating with wild-type organisms and selecting for homozygotes for the inserted marker. Next, the marker must be transferred to an appropriate background strain for the planned assay by mating. For example, in a RAS pathway assay for new pharmaceuticals one could perform the screen using a C. elegans lin-15 mutant that already contains a second or possibly a third detectable marker. In that case the positional marker pattern generated above would be transferred by mating into the lin-15 strain. If only fluorescent markers were going to be utilized, one can simply mix the different DNAs prior to insertion into the genome thus simultaneously adding all markers into the appropriate strain. The desired background is one that shows an optically detectable response to an active compound. This allows the organisms to be used to screen compound libraries for drug candidates. The marker pattern ensures that the detected signal is positionally correct for the screened activity. That is, it is quite likely that test compounds may have multiple activities that could result in positional changes in the expressed signal and/or anomalous expression. The marker pattern allows the system to discriminate between positional shifts in expression. As explained below, the pattern is especially effective in enabling detection of weak signals resulting from test compounds.

Using Marker Patterns To Detect Suppression of a Disease Model Phenotype

Certain disease model pathways involve the inappropriate activation of gene expression in certain tissues or in the migration of certain cell types during development of the animal (which then results in positional changes in marker expression). One such model involves the Wnt signaling pathway in C. elegans. Components of this pathway appear to be conserved in other organisms and have been shown to function in the development of several forms of cancer, including breast cancer (Nusse and Varmus, 1982; Lejeune et al., 1995) and colon cancer (Morin et al., 1997; Rubinfeld et al., 1997). Wnt signaling in C. elegans is involved in controlling the migration of specific cells (Korswagen et al., 2000). One example is that the proper migration of the QL neuroblast descendants depends upon the proper expression and function of the Wnt pathway genes mab-5 and egl-20 (Kenyon, 1986; Salser and Kenyon, 1992; Harris, et al., 1996). If the QL neuroblasts are marked with a fluorescent protein gene, the distance of these cells, which are normally located in the tail of the animal, from the vulva can be measured using marker pattern organisms. Note that the distance between the vulva signal and the M4 neuron signal in the pharynx allows for precise correction for the size of the animal and thus acts as an internal control. The disease model mutant displays inappropriate migration of these cells, or inappropriate expression of fluorescent protein in other cell types. In such a case, a high throughput drug discovery assay involves exposing the animals to compounds and determining which compounds caused the animals to assume a wild-type fluorescence pattern. The marker pattern enables the analysis to readily detect the shift of the positionally incorrect signal into a normal wild-type position. By allowing the signal analysis to focus on limited regions of the organism and/or by allowing a switching of the analysis mode (e.g., peak detection to integration) within specific (lineal) regions of the organism, the invention also allows the unambiguous detection of weak fluorescence signals.

Using Marker Patterns Recognition to Visualize Weak Signals

In some cases the autofluorescence (intrinsic fluorescence of the organism) signal of an organism is great enough to obscure the signal of a marker. In the case of C. elegans PY1089 GFP (Green Fluorescent Protein from Aequorea victoria) is expressed in two adjacent neurons in the head of the animal. That fluorescence is visible under the microscope as two areas of more concentrated green fluorescence in a background of diffuse autofluorescence. Current automated analytical instrumentation integrate the total fluorescence signal of the organism and are therefore not sensitive to the brighter region within the autofluorescence. Attempts to resolve this strain from wild type C. elegans using such automated systems have been unsuccessful thus far. When the electronic signal from the organism is monitored a clear peak signal is seen at one end of the animal. By synchronizing the signal analysis to a known marker pattern, it is possible to determine the orientation of the animal (e.g., head first) and analyze only the specific GFP signal from the head neurons.

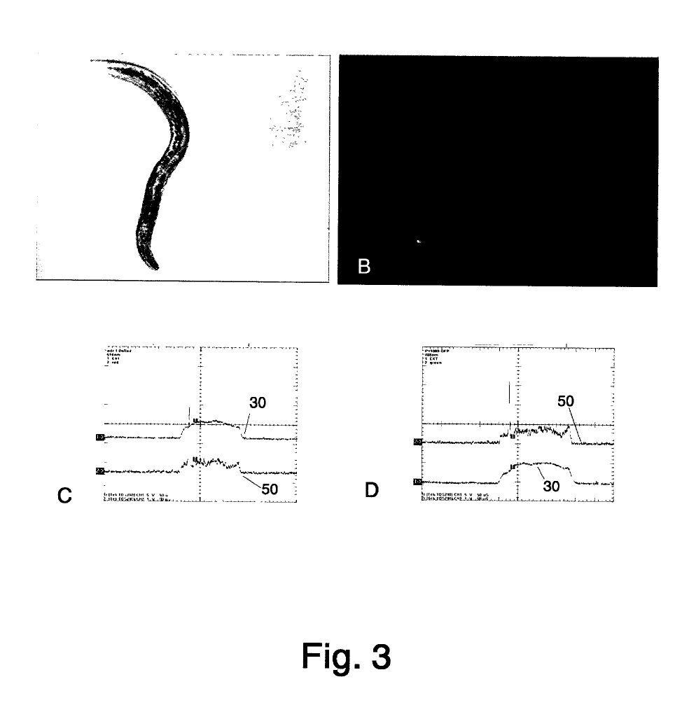

In the case of the animal whose oscilloscope traces are depicted in FIG. 3D, the total area under the fluorescence profile is 550 units while the area under the fluorescence peak is only 50 units resulting in a signal to noise ratio of 1:10. If, however, the area under the fluorescence peak is compared to an area of comparable width in the region of the animal with the highest autofluorescence that ratio changes to 2:1. If one then considers that the fluorescence signal from the two nerve cell bodies is 35 units, an assay is able to detect the presence of a third fluorescent cell body or the loss of one of the two fluorescent cell bodies.

A useful marker in this situation is the egl-17 positional markers described above. With egl-17::ZsYellow as the positional marker the instrument detects the M4 neuron in the anterior portion of the pharynx and the vulval precursor cells and rapidly determines the orientation of the animal as it passes through the analytical chamber. The software looks for the first green fluorescent peak immediately posterior to the M4 neuron and displays the intensity of only that signal. Results include signals such as 18 (no GFP fluorescence), 35 (GFP fluorescence in only one cell), 50 (fluorescence in two cells), 68 (3 cells), and 86 (4 cells).

FIG. 3 shows light and fluorescence photomicrographs of a C. elegans PY1089 animal are shown in FIG. 3A and FIG. 3B, respectively. Oscilloscope traces depicting the optical detector signals generated by two different PY1089 animals are shown in FIG. 3C and FIG. 3D. In FIG. 3B the fluorescence from the head neurons (lower end of imaged organism) is clearly visible and is distinguishable from the overall autofluorescence of the animal. Here the animal is oriented such that the two neurons are aligned one on top of the other and only one slightly diffuse spot is observed. In FIGS. 3C and 3D the one of the traces 30 shows the extinction signals from the animals while the other trace 50 show the green fluorescence signal. The animal in FIG. 3D was longer than the animal in FIG. 3C and was probably an adult. As expected, both the peak autofluorescence and the total autofluorescence (the area under the curve) are larger for the larger animal. The total fluorescence from the head neurons is approximately the same for both animals. For FIG. 3D the total area under the fluorescence curve is 542 units while the area under the GFP peak is 53. Of the peak area approximately 11.5 units are due to autofluorescence while the remaining 41.5 are due to the GFP fluorescence. These measurements indicate that discrimination could be made automatically provided that a marker pattern is available to automate decisions concerning the organism's orientation.

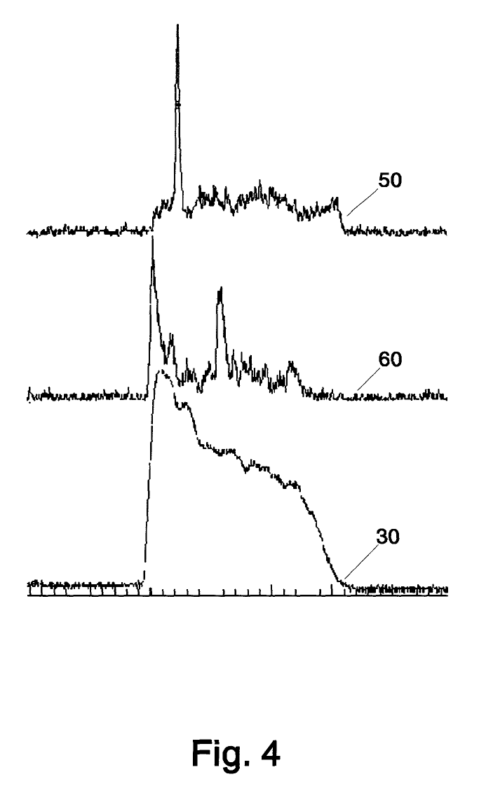

FIG. 4 is a graphic representation (oscilloscope) of the optical detector signals from an egl-17::Zs Yellow construct mated into PY1089. The presence of an organism is determined by an extinction signal 30. A yellow fluorescence signal 60 (ZsYellow) clearly marks the head end of the organism (sharp spike used by the software to determine orientation) and provides several other fluorescence peaks along the length of the organism. A more diffuse green fluorescence signal 40 (GFP) is then integrated to determine the head neurons, which immediately follow the yellow M4 neuron peak.

The various marker patterns provided by the present invention allow the software to determine the orientation of elongate organisms, allow the software to specifically measure the position of treatment dependent signals (by comparison to invariant marker pattern signals), allow the software to alter the mode of signal analysis (e.g. peak detection versus signal integration) in a positionally controlled manner, and allow the software to limit detailed data analysis to specific positions along the length of the test organism. From the forgoing description a number of uses of the marker pattern organisms will be apparent to those of skill in the art. One method is to produce a test organism that expresses a marker pattern and also variably displays a detectable signal in response to one or more treatments. Generally a treatment will be exposure of the test organism to one or more test compounds, for example, to select active drug candidates from a synthesis library. However, the treatment may also include one or more environmental or other factors that potentiate or otherwise affect the action of the test compound. After the exposure to the treatment, the test organism is analyzed by a flow cytometer. The marker pattern is detected and the analytic software of the system uses the marker pattern to effectively analyze the signal that represents treatment response. As explained above, such analysis would be impossible or much less efficient without use of the marker pattern. It will be appreciated that a major goal is to select out organisms on the basis of their response to the treatment. This requires that data analysis be completed before the organism passes through the sorting section of the flow cytometer. Therefore, data analysis time is very brief and the enhanced analysis permitted by the use of marker patterns is often crucial.

The following claims are thus to be understood to include what is specifically illustrated and described above, what is conceptually equivalent, what can be obviously substituted and also what incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

### REFERENCES

Burdine, et al., “egl-17 encodes an invertebrate fibroblast growth factor family member required specifically for sex myoblast migration in Caenorhabditis elegans,” Proc. Natl. Acad. Sci., U.S.A. 94:2433-2437.

Burdine, et al., “EGL-17 (FGF) expression coordinates the attraction of the migrating sex myoblasts with vulval induction in C. elegans,” Development 125:1083-1093.

Byerly, L., R C. Cassada and R L. Russell. Machine for rapidly counting and measuring the size of small nematodes. Rev Sci Instrum. 1975 May; 46(5):517-22.

Harris, J., Honigberg, L., Robinson, N., and Kenyon, C. (1996). Neuronal cell migration in C. elegans: regulation of Hox gene expression and cell position. Development, 122, 3117-31.

Kenyon, C. (1986). A gene involved in the development of the posterior body region of C. elegans. Cell, 46, 477-487.

Korswagen, H. C., Herman, M. A., and Clevers, H. C. (2000). Specificity of β-catenin signalling in C. elegans. Nature, in press.

Lejeune, S., Huguet, E. L., Hamby, A., Poulsom, R., and Harris, A. L. (1995). Wnt5a cloning, expression, and up-regulation in human primary breast cancers. Clin Cancer Res, 1, 215-22.

Morin, P. J., Sparks, A. B., Korinek, V., Barker, N., Clevers, H., Vogelstein, B., and Kinzler, K. W. (1997). Activation of beta-catenin-Tcf signaling in colon cancer by mutations in beta-catenin or APC. Science, 275, 1787-90.

Nusse, R., and Varmus, H. E. (1982). Many tumors induced by the mouse mammary tumor virus contain a provirus integrated in the same region of the host genome. Cell, 31, 99-109.

Rubinfeld, B., Robbins, P., El-Gamil, M., Albert, I., Porfiri, E., and Polakis, P. (1997). Stabilization of beta-catenin by genetic defects in melanoma cell lines. Science, 275, 1790-2.

Salser, S. J., and Kenyon, C. (1992). Activation of a C. elegans Antennapedia homologue in migrating cells controls their direction of migration. Nature, 355, 255-8.

Tsien, R. (1999). Rosy Dawn for Fluorescent proteins. Nature Biotechnology, 17: 956-57.

What is claimed is:

1. A process for analyzing elongate multicellular organisms by flow cytometry comprising the steps of:
creating a population of test organisms wherein each member of the population displays a marker pattern, said marker pattern representing a plurality of spatially consistent first features spaced apart along a length of each organism and wherein each member of the population also displays at least one of a second feature modifiable or inducible when the population is subjected to a test treatment, each of said first and said second features being detectable through analysis with a flow cytometer;
subjecting the population to a test treatment;
analyzing members of the population with a flow cytometer equipped to process elongate multicellular organisms;
detecting the marker pattern on the members analyzed; and
using the detected marker pattern to determine status of the second feature on each of the members analyzed.

2. The process according to claim 1, wherein the step of creating a population includes the step of producing a transgenic organism.

3. The process according to claim 2, wherein the step of producing a transgenic organism includes choice of a particular promoter.

4. The process according to claim 1, wherein the marker pattern is detectable by a flow cytometer by use of detection means selected from the group consisting of light scatter, light absorption and fluorescence.

5. The process according to claim 1, wherein the step of subjecting the population to a test treatment includes contacting the population with a candidate drug molecule.

6. The process according to claim 1, wherein the second feature responds to the test treatment by a change detected as an optical signal, the change being one selected from the group consisting of an increased signal, a decreased signal or a positionally altered signal.

7. The process according to claim 1, wherein the step of using the detected marker pattern includes the step of determining a longitudinal orientation of each member of the population analyzed.

8. The process according to claim 1, wherein the step of using the detected marker pattern includes the step of limiting analysis of data corresponding to the second feature to a particular longitudinal region of each of the members analyzed.

9. The process according to claim 1, wherein the step of using the detected marker pattern includes the step of altering a mode data analysis for data corresponding to the second feature in a particular longitudinal region of each of the members analyzed.

10. The process according to claim 9, wherein the mode of data analysis is selected from the group consisting of signal peak analysis and signal integration.

11. A process for preparing a model strain of elongate multicellular organisms intended for specialized flow cytometry analysis comprising the steps of:
creating a marker strain of organisms wherein each member of the strain displays a marker pattern, said marker pattern representing a plurality of marker features spaced apart along a length of each organism and spatially consistent from member to member, said marker features being detectable through analysis with a flow cytometer;
creating a test strain of organisms wherein each organism of the test strain displays at least one test feature modifiable or inducible when the test strain is subjected to a test treatment, said test features being detectable through analysis with a flow cytometer; and
creating a model strain by combining the marker pattern from the marker strain with the test features from the test strain so that each organism of the model strain displays both the marker pattern and at least one test feature.

12. An organism belonging to a model strain produced by the process of claim 11.

13. A process for analyzing elongate multicellular organisms by flow cytometry comprising the steps of:
subjecting a population of the model strain of claim 11 to a test treatment;
analyzing members of the subjected population with a flow cytometer equipped to process elongate multicellular organisms;
detecting the marker pattern on the members analyzed; and
using the detected marker pattern to determine status of the test feature on each of the members analyzed.

14. The process according to claim 11, wherein the step of creating a population includes the step of producing a transgenic organism.

15. The process according to claim 14, wherein the step of producing a transgenic organism includes choice of a particular promoter.

16. The process according to claim 13, wherein the marker pattern is detectable by a flow cytometer by use of detection means selected from the group consisting of light scatter, light absorption and fluorescence.

17. The process according to claim 13, wherein the step of subjecting the population to a test treatment includes contacting the population with a candidate drug molecule.

18. The process according to claim 13, wherein the test feature responds to the test treatment by a change detected as an optical signal, the change being one selected from the group consisting of an increased signal, a decreased signal or a positionally altered signal.

19. The process according to claim 13, wherein the step of using the detected marker pattern includes the step of determining a longitudinal orientation of each member of the population analyzed.

20. The process according to claim 13, wherein the step of using the detected marker pattern includes the step of limiting analysis of data corresponding to the second feature to a particular longitudinal region of each of the members analyzed.

21. The process according to claim 13, wherein the step of using the detected marker pattern includes the step of altering a mode data analysis for data corresponding to the second feature in a particular longitudinal region of each of the members analyzed.

22. The process according to claim 21, wherein the mode of data analysis is selected from the group consisting of signal peak analysis and signal integration.
